(19) United States
(12) Patent Application Publication
(10) Pub. No.: US 2026/0314909 A1
(43) Pub. Date: Oct. 15, 2026

(54) Transmitting enhanced images of tissue and structures from inside the body

(71) Applicant: AI Lumen LLC, Austin, TX (US)

(72) Inventors: Michael Calhoun, Lighthouse Point, FL (US); Gary Shymkiw, Austin, TX (US); William Turner, Abingdon, MA (US); Benjamin Wilson, Falmouth, ME (US)

(73) Assignee: AI Lumen LLC, Austin, TX (US)

(21) Appl. No.: 19/636,454

(22) Filed: Apr. 1, 2026

Related U.S. Application Data
(60) Provisional application No. 63/781,967, filed on Apr. 1, 2025.

(51) Int. Cl. A61B 1/00; A61B 1/04; A61B 90/00; G06T 3/4046; G06T 3/4053; G06T 5/60; G06T 5/80; H04N 7/18

(52) U.S. Cl. CPC A61B 1/00016; A61B 1/000095; A61B 1/000096; A61B 1/0005; A61B 1/041; G06T 3/4046; G06T 3/4053; G06T 5/60; G06T 5/80; H04N 7/181; A61B 2090/367; A61B 2090/371; G06T 2207/10016; G06T 2207/10068; G06T 2207/20081; G06T 2207/20084; G06T 2207/30004

(57) ABSTRACT

Relatively low-resolution image data is transmitted from inside the body to outside the body. An image enhancer examines the relatively low-resolution image data, removes distortion, and upscales that image data to a higher resolution sufficient for medical personnel to identify and treat medical conditions within the patient. The image enhancer provides undistorted and higher resolution image data, having been configured in response to simulated distortion and downsampling of a set of ground truth higher resolution image data.

### COPYRIGHT NOTICE

A portion of the disclosure of this patent document contains material subject to copyright protection. The copyright owner has no objection to the facsimile reproduction by anyone of the patent document or the patent disclosure as it appears in the official Patent and Trademark Office patent file or records, but otherwise reserves all copyright rights whatsoever.

### BACKGROUND

This background is provided as a convenience to the reader and does not admit to any prior art or restrict the scope or spirit of the disclosure or the invention. This background is intended as an introduction to the general nature of technology to which the disclosure or the invention can be applied.

### FIELD OF THE DISCLOSURE.

This Application generally relates to methods and systems for viewing structures in the body, for presenting images of those structures to medical personnel, for performing medical procedures with respect to those structures using those images. Apparatus and methods described herein can be used with respect to viewing tissue near an endoscope, the endoscope having been inserted into the body and manipulated to a location that might be of interest. One circumstance in which the apparatus and methods described herein can be of use is when viewing internal body tissue and/or structures, such as possible polyps, in the course of a colonoscopy, and possibly manipulating those internal body structures. While this Application primarily describes use with internal body structures with respect to a colonoscopy, apparatus and methods described herein can be used with respect to virtually any tissues or structures internal to the body.

### RELATED ART

One medical technique for investigating and possibly preventing colorectal cancer is to perform a medical procedure known as a colonoscopy. This procedure generically involves inserting an endoscope into the body and viewing tissues or other body structures using a camera coupled to a distal end of the endoscope. Images, such as real-time images of those tissues or structures, can be presented to medical personnel on a monitor or other video display. Medical personnel can manipulate the endoscope to direct the camera for viewing selected tissues or structures in the colon, such as to evaluate whether those tissues might be can-cerous or otherwise deleterious, and to take action with respect to those tissues (such as administering medication or performing surgery with respect thereto).

One problem that has arisen in the art is that tissues or other body structures might be situated in locations where they are hidden from view from the endoscope. For example, tissues or structures of interest might be disposed behind or within folds in the colon, so that those tissues or structures of interest are hidden behind or within structures that have the effect of blocking a view from the endoscope. When this occurs, cameras coupled to the endoscope can be generally unable to obtain images of those tissues or to present those images to medical personnel. This can pose a problem with respect to a relatively complete investigation of those tissues or structures of interest, even when those tissues or structures are otherwise within or near a range of operation of the endoscope.

One known technique is to provide a mechanical device that is capable of adjusting the endoscope to direct its camera to receive an image from the location of interest. For example, a distal element of the endoscope can be retroflexed to dispose a camera coupled to that distal element in a rearward direction. In such cases, medical personnel performing the procedure would manipulate the endoscope to perform retroflexing and thus direct the camera in a rearward direction (or to otherwise direct the camera to view the tissues or structures of interest).

While this technique can sometimes promote the goal of allowing medical personnel to view tissues or structures of interest that might otherwise be difficult to view, it can be subject to one or more drawbacks. For one example, manipulating the retroflexing elements can be complex and can take extra time. Medical personnel might have only a limited amount of time in which to perform the colonoscopy, which could reduce the effectiveness of that procedure and possibly require additional intervention at another time.

For another example, when the retroflexing elements are moved within the body, there might be additional risk of damage to tissues or structures internal to the body, such as due to movement of the retroflexing elements near sensitive tissues or structures, which could increase the risk of the procedure to the patient. Due to the required radius necessary for retroflexion. it is also possible for a colonoscope disposed in a retroflexed orientation to become stuck in the colon as the radius of the colonoscope may be equivalent or may exceed the diameter of the colon; in this example, it can be difficult to remove the colonoscope and also might pose a medical risk to the patient.

One known technique is to provide a second, third, or other additional camera disposed to receive images in a second direction, such as possibly in a perpendicular direction from the endoscope. For a first example, the endoscope can be replaced with a new endoscope that has one or more additional cameras disposed to receive images in a perpendicular direction. For a second example, an accessory for the endoscope can include one or more additional cameras that can be coupled to the endoscope and which is disposed to receive images in a perpendicular direction.

While this technique can sometimes promote the goal of allowing medical personnel to view tissues or structures of interest that might otherwise be difficult to view, it can be subject to one or more drawbacks. For one example, adding additional cameras to the endoscope can alter the functionality of the endoscope. Known endoscopes in current use are believed to be well entrenched in the market for medical devices and are often considered foundational to their associated medical procedure. Physicians are often accustomed to and have preferences for the way particular endoscopes perform. Altering endoscope design to accommodate additional cameras and their supporting devices might alter the basic functionality of the endoscope, at least in terms of manipulation, access to the working channel of the endoscope, flushing/inflation, and possibly other performance characteristics. Many physicians are not willing to accept those changes.

Accordingly, it would be advantageous to provide apparatus and methods disposed to allow medical personnel to view tissues and structures internal to the body from outside the body. It would be advantageous to provide apparatus and methods disposed to wirelessly transmit and to present images from inside the body to outside the body, at sufficiently high resolution, and in sufficiently near real-time, that medical personnel can use details from those images with one or more medical procedures.

### SUMMARY OF THE DISCLOSURE

This summary of the disclosure is provided as a convenience to the reader and does not limit or restrict the scope or spirit of the disclosure or the invention. This summary is intended as an introduction to more detailed description found in this Application, and as an overview of techniques explained in this Application. The described techniques have applicability in other fields and beyond the embodiments specifically reviewed in detail.

In one embodiment, an endoscope is fitted with a relatively thin accessory having a rearward-facing camera. In one embodiment, the rearward-facing camera can be disposed so that medical personnel can select images for display from the endoscope's forward-facing camera, or by selection, from the accessory's rearward-facing camera. This can have the effect that medical personnel have their choice of which direction to look from the endoscope within the patient.

While this Application primarily describes a system in which the rearward-facing camera is disposed at a substantial angle from the forward-facing camera, such as possibly directed at images in a direction 180° different from the forward-facing camera (or otherwise), there is no particular requirement for any such limitation. After reviewing this Application, those skilled in the art would recognize that many other configurations and/or angles for the rearward-facing camera would be workable, and are within the scope and spirit of the invention.

In one embodiment, the rearward-facing camera is disposed to transmit wireless signals including image data at a relatively low resolution. The relatively low resolution can have the effect that the rearward-facing camera can provide a relatively smooth image (whether in a sequence of still images or in a video image) and does not interfere with perception by medical personnel performing the medical procedure.

In one embodiment, the relatively low resolution image data is received from inside the body at a location outside the body. An image enhancer is disposed to examine the relatively low resolution image data and to upscale that image data to a higher resolution sufficient for medical personnel to identify and treat medical conditions within the patient.

In one embodiment, the image enhancer is disposed to determine when the relatively low resolution image data includes one or more recognizable image snippets. When those recognizable image snippets are identified, the image enhancer can retrieve higher resolution image data and improve the received image data using that higher resolution image data. For example, the image enhancer can improve the received image data by replacing the identified image snippets with the higher resolution image data. This can have the effect that medical personnel can perform the medical procedure as if they were able to directly receive image data from inside the body at the higher resolution.

Accordingly, apparatus and methods can be disposed to provide medical personnel with image data from inside the body that is sufficiently real-time that medical personnel do not perceive delay or distortion in presentation of images from inside the body. When that image data is presented to medical personnel, it can be presented at a sufficiently high resolution that medical personnel can identify fine-grained information about the image, so as to identify tissues and structures within the body that should receive more attention.

After reading this Application, those skilled in the art would understand how systems such as those described herein can be used to perform functions described herein to achieve desired results.

### BRIEF DESCRIPTION OF THE FIGURES

In the figures, like references generally indicate similar elements, although this is not strictly required.

FIG. 1 shows a conceptual drawing of an example system.

FIG. 2 shows a conceptual drawing of an example endoscope.

FIG. 3 shows a conceptual drawing of an example method of using the system.

FIG. 4 shows a conceptual drawing of an example image enhancer.

After reading this Application, those skilled in the art would recognize that the figures are not necessarily drawn using any particular formalism or rubric, nor are they necessarily drawn to scale for construction, nor do they necessarily specify any particular location or order of construction.

### DETAILED DESCRIPTION

### Figures and Text

### Example system

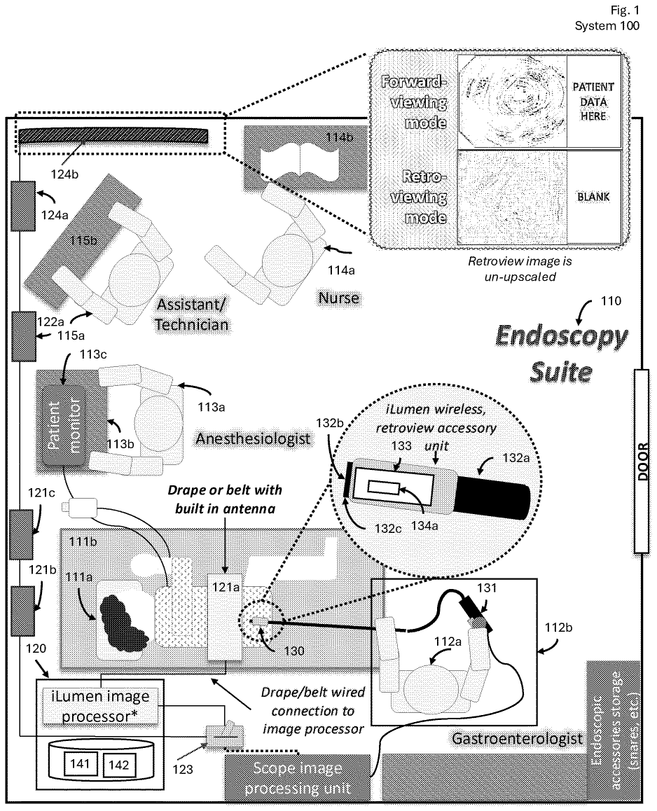

FIG. 1 shows a conceptual drawing of an example system.

In one embodiment, an example system 100 is described herein with respect to an endoscopy suite 110 in which a medical procedure such as an endoscopy is capable of being performed. The endoscopy suite 110 can define a medically clean region within which medical personnel can perform a selected medical procedure with respect to a patient 111a. For an example medical procedure such as an endoscopy, the patient 111a can be situated on a patient bed 111b.

In one embodiment, the endoscopy suite 110 can be disposed for use by medical personnel including a gastroenterologist 112a, and can include a gastroenterologist workstation 112b available, including an anesthesiologist 113a, such as including anesthesiologist workstation 113b available, and optionally including one or more additional medical personnel such as a nurse 114a using a nurse's workstation 114b, an assistant/technician 115a using an assistant/technician workstation 115b, or otherwise as described herein. The endoscopy suite 110 can also include an image processing system 120 such as an example described herein, and possibly other medical equipment and/or systems associated with medical procedures.

As described herein, the gastroenterologist workstation 112b can be disposed to assist the gastroenterologist 112a in performing the medical procedure, and can include at least an endoscope controller 131 and an image selection switch 123. The gastroenterologist 112a can be positioned so as to have a clear view of a display device 124b, and can have one or more additional display devices or similar elements disposed at the gastroenterologist workstation 112b for their use.

As described herein, the anesthesiologist workstation 113b can be disposed to assist the anesthesiologist 113a in performing the medical procedure, and can include at least a patient monitor 113c coupled to the patient 111a. The anesthesiologist workstation 113b can include one or more additional display devices or similar elements disposed for the anesthesiologist 113a to monitor the patient 111a.

Similarly, the nurse's workstation 114b and the assistant/technician's workstation 115b can be disposed to assist those medical personnel with their roles in performing the medical procedure, and can include control elements and/or display elements suitable for those roles.

### Image Processing System

In one embodiment, the image processing system 120 can include an endoscope 130 such as an example described herein. The image processing system 120 can include an image receiver 121b disposed to receive image data from the endoscope 130 and an image postprocessor 121c disposed to postprocess that image data, such as described herein. The image processing system 120 can include an image enhancer 122a, such as described herein. The image processing system 120 can also include an image selection switch 123 operable by the gastroenterologist 112a or at his direction, and a display processor 124a and a display device 124b disposed to display images to the gastroenterologist 112a or to an assistant, and optionally other elements as described herein.

As described herein, the image processing system 120 can obtain image data from inside the patient 111a, such as from an endoscope capsule 132c disposed at a distal end of the endoscope 130. The image processing system 120 can enhance image data using the image enhancer 122a. The image processing system 120 can present enhanced image data to medical personnel using one or more display devices.

### Example Endoscope

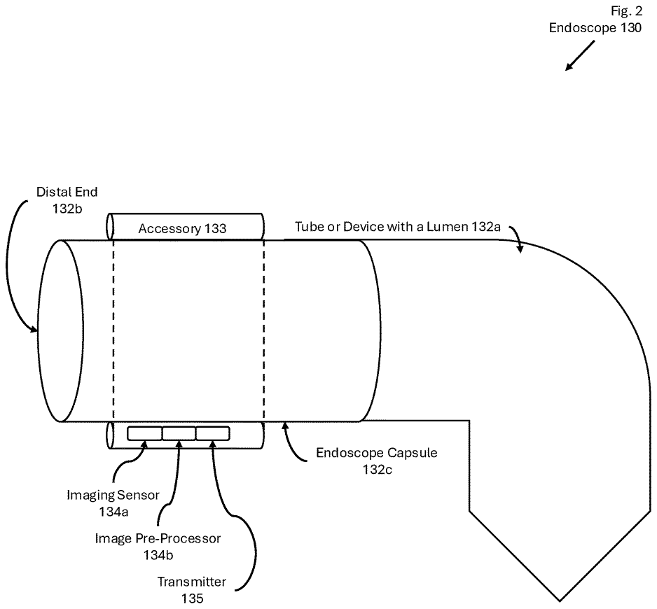

FIG. 2 shows a conceptual drawing of an example endoscope.

In one embodiment, the endoscope 130 can be couplable to an endoscope controller 131. The endoscope controller 131 can be disposed to be available for the gastroenterologist 112a to control the endoscope 130.

In one embodiment, the endoscope 130 can include a tube or device having a lumen 132a and having a distal end 132b, an operational channel, a set of control connectors, a set of substance transfer channels, and an endoscope capsule 132c. The endoscope capsule 132c can be disposed at the distal end 132b and can include one or more elements disposed to perform functions associated with endoscopy. For example, the endoscope capsule 132c can include one or more cameras or other sensors, can include one or more devices for manipulating tissue or other structures internal to the body (such as for ablating or removing tissue, dispensing medication, obtaining or testing samples, or otherwise associated with endoscopy), or otherwise as described herein.

In one embodiment, the endoscope 130 can also include an accessory 133, such as couplable to the distal end 132b. The accessory 133 can include an imaging sensor 134a, such as a camera or an ultrasound transceiver. The camera can be disposed to receive image data at one or more frequencies, such as one or more visible frequencies, one or more infrared frequencies, one or more ultrasonic frequencies, one or more other frequencies with which data can be encoded, or otherwise as described herein.

While this Application primarily describes image data as being encoded using a frequency (such as a carrier frequency for modulation), in the context of the invention there is no particular requirement for any such limitation. After reading this Application, those skilled in the art would recognize that many alternatives would be workable and are within the scope and spirit of the invention. For example, image data can be encoded using pulse code modulation or another type of modulation or transmission from within the body to outside the body. The system can be configured and disposed to enhance image data (such as possibly by undoing distortion or other artifacts of capture and/or transmission, or such as possibly by upscaling the image from the fineness of resolution available for practical transmission from within the body to outside the body), or otherwise as described herein.

While this Application primarily describes the imaging sensor 134a as operating using electromagnetic frequencies, in the context of the invention there is no particular requirement for any such limitation. For one example, the imaging sensor 134a can be disposed to obtain image data in response to electromagnetic frequencies (whether visible, non-visible, or a combination or conjunction thereof), other electromagnetic frequencies such as radio or x-rays, ultrasound, or magnetic coupling. For another example, the imaging sensor 134a can be disposed to obtain image data in response to measures of pressure, chemical absence/presence, or otherwise as described herein.

In one embodiment, the endoscope 130 can also include an image pre-processor 134b, such as disposed to manipulate image data before transmitting that image data from inside the body. For example, manipulating image data can include (a) compressing that image data, (b) encrypting or obfuscating that image data, such as to provide security against data leakage, (c) formatting that image data into one or more data packets for transmission.

In one embodiment, the imaging sensor 134a and the image preprocessor 134b can include one or more of the following devices or implement one or more of the following techniques:
For example, the imaging sensor 134a can include an OVM6946 chip disposed to obtain image data, or another chip with similar functionality. For example, the imaging sensor 134a can additionally or alternatively include an OV7676 chip disposed to obtain image data.
For example, the image preprocessor 134b can compress image data using H.264, H.265, JPEG, or another technique.
For example, the image preprocessor 134b can encrypt image data using AES-128, AES-256, or another technique.
For example, the image preprocessor 134b can format image data into 4096-byte (of data) packets, or another packet format.

In one embodiment, the endoscope 130 can also include a transmitter 135, such as disposed to emit information associated with the image data for receipt by the image receiver 121b. For example, the transmitter 135 can transmit image data at about 2 Mbps and a frame rate of about 15-30 frames per second, using a transmission frequency of about 434 MHz. In an alternative embodiment, the transmitter 135 can in addition or instead transmit image data using a transmission frequency of about 915 MHz, or another frequency associated with the transmitter 135. Other data transmission rates and frequencies would of course be workable and are within the scope and spirit of the invention.

### Example Method of Use

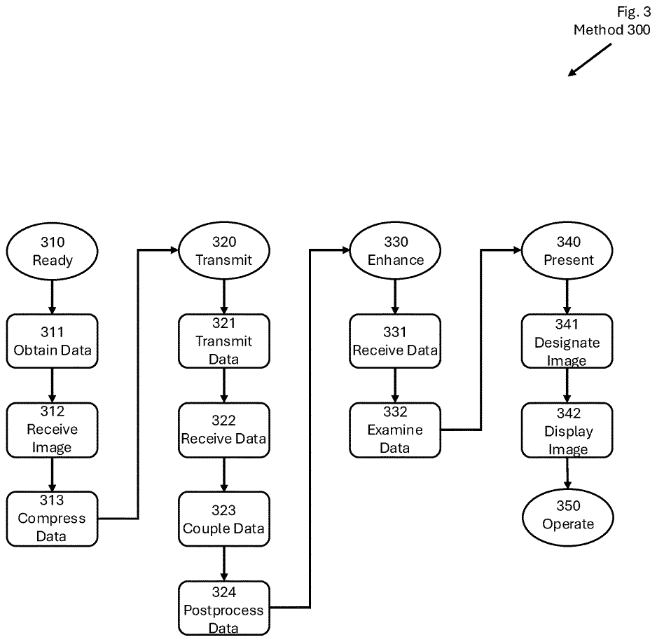

FIG. 3 shows a conceptual drawing of an example method of using the system.

A method 300 of using the system 100 is described herein with respect to particular flow points and method steps, such as shown in the figure or described herein. By the nature of the written word, these flow points and method steps are described in a particular order; however, this description does not limit the method to this particular order or any other particular order. These flow points or method steps can be performed in a different order; concurrently or partially concurrently; otherwise in a parallel, pipelined, quasi-parallel manner; or in another manner. These flow points or method steps can be performed in part, paused, and returned to for completion. These flow points or method steps can be performed as co-routines or otherwise. In the context of the invention, there is no particular reason for any of these limitations.

One or more portions of the method 300 are sometimes described as being performed by particular elements of the system, as described with respect to the figures, or sometimes by “the method” itself. When a flow point or method step is described as being performed by “the method,” it can be performed by one or more of those elements, by one or more portions of those elements, by an element not described with respect to the figure, by a combination or conjunction thereof, or otherwise. For example, flow points or method steps can be performed either by general-purpose hardware or special-purpose hardware, such as ASICs or other custom chips or specialized circuitry.

This Application primarily describes one possible implementation, in which one or more elements of the system include programmable computing devices interacting with other elements. However, there is no particular requirement for any of these limitations. For example, the same or similar effects can be achieved using one or more electronic circuits, one or more specialized processing devices operating using specialized control elements, one or more remote computing devices directing operation of one or more of the sensing and operating devices described, or otherwise to achieve the same or similar effects as described herein.

In one embodiment, the method 300 is described with respect to an example medical procedure, such as a colonoscopy. In such cases, as described herein, the method 300 is started at a first defined step within the medical procedure and finished at a second defined step within the medical procedure. Thus, as described herein, the method 300 is performed while the medical procedure is ongoing. While this Application primarily describes the method 300 as being performed in a repeated loop while the medical procedure is ongoing, there is no particular requirement therefore. For example, the method 300 can be started, stopped, restarted, paused, or otherwise, in response to actions by medical personnel, whether before, during, or after any one medical procedure.

### Ready to Collect Image Data

A flow point 310 indicates that the method 300 is ready to collect image data from inside the body.

At a step 311, the imaging sensor 134a obtains image data from tissue or structures internal to the body. For example, the imaging sensor 134a can obtain imaging data associated with one or more frequencies of visible light, one or more frequencies of energy in a non-visible portion of the electromagnetic spectrum, one or more frequencies of ultrasound, or otherwise as described herein. While this Application primarily describes the imaging sensor 134a as “obtaining” image data, there is no particular requirement for any such limitation. For example, the imaging sensor 134a can receive or generate image data, such as in response to one or more data stores or devices from which image data can be derived, generated, augmented, filtered, or modified.

At a step 312, the image pre-processor 134b can receive image data from the imaging sensor 134a and makes ready to pre-process that image data for transmission from inside the body. In one embodiment, the image preprocessor 134b can be configured to prepare the image data for transmission from inside the body to a receiver outside the body.

At a step 313, the image preprocessor 134b can compress, encrypt/obfuscate, and format image data for transmission. As described herein, the image preprocessor 134b (a) can compress image data using H.264, H.265, JPEG, or another technique, (b) can encrypt image data using AES-128, AES-256, or another technique, and (c) can format image data into 4096-byte (of data) packets, or another packet format.

### Ready to Transmit Image Data

A flow point 320 indicates that the method 300 is ready to transmit image data from inside the body.

At a step 321, the transmitter 135 can transmit image data at about 2 Mbps using a frequency of about 434 MHz.

At a step 322, the image receiver 121b can receive image data sent by the transmitter 135 from inside the body. The image receiver 121b can include an antenna 121a disposed near the transmitter 135, such as on a drape or belt on or near the exterior of the patient 111a (such as possibly coupled to a gown or other clothing worn by, draped over, or placed near the patient 111a).

At a step 323, the image receiver 121b can couple image data sent from inside the body to the image postprocessor 121c.

At a step 324, the image postprocessor 121c can postprocess image data sent from inside the body. In one embodiment, the image postprocessor 121c can (a) collect image data from multiple packets into a unified format, (b) decrypt and/or deobfuscate image data, such as using an AES-128, AES-256, or another decryption technique, or a deobfuscation technique, or a combination or conjunction thereof, (c) decompress image data, such as using H.264, H.265, JPEG, or another technique, and (d) couple image data to the image enhancer 122a.

### Ready to Enhance Image Data

A flow point 330 indicates that the method 300 is ready to enhance image data from inside the body.

At a step 331, the image enhancer 122a can receive image data from the image postprocessor 121c.

At a step 332, the image enhancer 122a can examine image data for one or more images 141. In one embodiment, each image 141 can represent at least a portion of an image, such as an image that might be received or generated from inside the body by the imaging sensor 134a. For one example, each image 141 might represent tissue or structures in the body as viewed from one selected direction. For another example, each image 141 might represent tissue or structures in the body as viewed with only limited resolution.

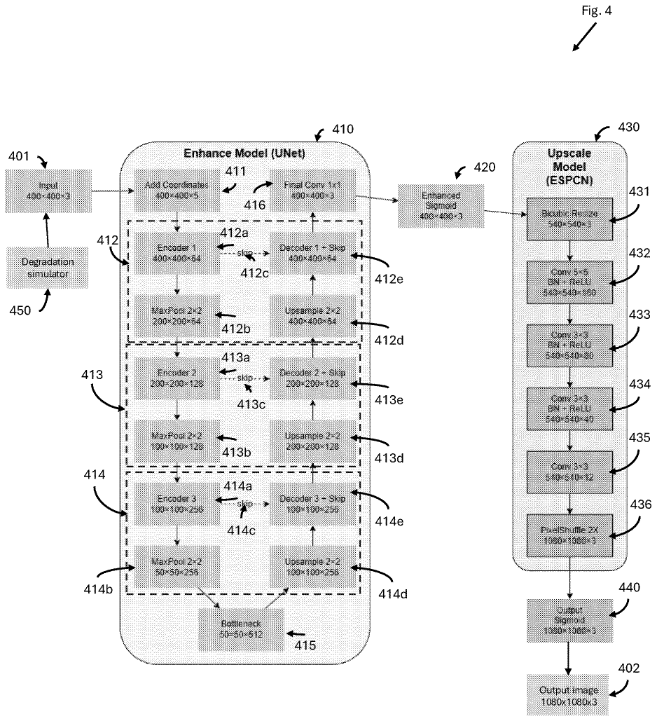

In one embodiment, the image enhancer 122a can use one or more artificial intelligence or machine learning techniques, to make its determination of replacing or improving image data using enhanced image data 142. In one embodiment, the image enhancer 122a can operate as described with respect to FIG. 4.

For example, as described herein, the image enhancer 122a can use one or more of the following artificial intelligence or machine learning techniques:
ESRGAN (enhanced super-resolution generative adversarial networks),
SRCNN (super-resolution convolutional neural network),
FSRCNN (fast super-resolution convolutional neural network),
VDSR (very deep super resolution),
SwinIR (image restoration software using a Swin transformer),
ESPCN (efficient sub-pixel convolutional neural network), or otherwise as described herein.

In one embodiment, the enhanced image data 142 can represent the “best approximation” at higher resolution of image data associated with the tissue or structures inside the body. The image enhancer 122a can substitute the enhanced image data 142 for the one or more images 141 in at least some portion of the received image data.

For example, the images 141 can represent image data at a resolution of about 400 pixels x 400 pixels per frame. Limiting image data to about this resolution can allow the imaging sensor 134a and image preprocessor 134b to prepare image data for transmission by the transmitter 135 at about 30 frames/second with a lag of no more than about 200 msec. This can have the effect that the gastroenterologist 112a is able to view the tissue or structure inside the body at sufficiently close to real-time such that they do not perceive the image/video data as delayed, distorted or otherwise perceivably different from images as supplied by the forward view native camera.

### Ready to Approximate a Display Resolution

A flow point 340 indicates that the method 300 is ready to present image data to medical personnel, such as to the gastroenterologist 112a or to an assistant.

At a step 341, the system 100 can determine which image data the gastroenterologist 112a (or an assistant) has designated for display using the image selection switch 123. For example, the gastroenterologist 112a might designate the ordinary forward image, from the forward camera in the endoscope capsule 132c, or the rearward image, from the accessory 133, for display. The system 100 collects the image data selected by the gastroenterologist 112a and couples that selected image data to the display processor 124a.

At a step 342, the display processor 124a processes the selected image data for presentation on the display device 124b. In one embodiment, the image data can represent still images, video, or some combination or conjunction thereof. For example, the image data can represent still images at which the gastroenterologist 112a desires to look more closely. For another example, video data can represent video images with respect to gradient changes in conditions of the patient's tissue or structures.

While this Application primarily describes only one display processor 124a and only one associated display device 124b, in the context of the invention there is no particular reason for any such limitation. For a first example, one display processor 124a can be coupled to multiple display devices 124b and can present part or all of the selected image data on one or more of those multiple display devices 124b. For a second example, multiple display processors 124a can be coupled to multiple portions of the image data and can present part or all of the selected image data on one or more of those multiple display devices 124b.

Also, while this Application primarily describes presenting only the selected image data on a display device 124b, in the context of the invention there is no particular reason for any such limitation. For an alternative example, a first display processor 124a can be coupled to the selected image data, so as to present that selected image data on an associated display device 124b. A second display processor 124a can be coupled to the non-selected image data, so as to present that non-selected image data on a different display device 124b.

For another alternative example, each one of a plurality of display processors 124a can each determine whether to present the forward image from the endoscope capsule 132c or the rearward image from the accessory 133, such as in response to a separate image selection switch 123. This can have the effect that the gastroenterologist 112a and an assistant can each separately determine which such image data to present for their own viewing, independent of each other.

In one embodiment, the endoscope 130 can be disposed with a first camera integrated therein and a second camera associated therewith integrated with the endoscope capsule 132c. However, there is no particular requirement for any such limitation; after reviewing this Application, those skilled in the art would recognize that many alternatives would be workable and are within the scope and spirit of the invention.

Having selected which image to present on the display device 124b, the gastroenterologist 112a (or an assistant) can operate the image selection switch 123 to select the forward camera associated with the endoscope capsule 132c or the rearward camera associated with the accessory 133.

### Selecting Forward or Rearward Camera

While this Application primarily refers to the image selection switch 123 as a switch, in the context of the invention there is no particular reason for any such limitation. For example, the image selection switch 123 can be any status element responsive to one or more devices coupled to the system 100 (such as the gastroenterologist workstation 112b). The gastroenterologist 112a (or an assistant) can manipulate the image selection switch 123 using touch controls operable by hand (such as a button or lever) or foot (such as a pedal), by eye gesture or hand gesture (such as using an eye tracking technique or gesture tracking technique), by voice (such as using voice recognition), or as otherwise known in the art of user interfaces.

For another example, the image selection switch 123 can be disposed to operate on a timer, that is, the gastroenterologist 112a might designate a selected image to be displayed only momentarily, such as for a few seconds (for example, 30 seconds or less), to be displayed only so long as the endoscope 130 is not moved within the patient 111a, or otherwise as described herein.

### Controlling the Endoscope

A flow point 350 indicates that the gastroenterologist 112a is ready to operate the endoscope controller 131. This can have the effect that that the gastroenterologist 112a can control the endoscope 130, such as to
(a) move the endoscope 130 within the body of the patient 111a, such as to move the endoscope 130 within the patient's colon,
(b) operate controls for the endoscope 130, such as to alter operating parameters for the imaging sensor 134a, apply medication or other substances, collect sample tissue or structures, ablate malign tissue or structures, or perform other procedures with respect to tissue or structures inside the body of the patient 111a,
or otherwise as described herein.

### Example Image Enhancer

FIG. 4 shows a conceptual drawing of an example image enhancing system.

The image enhancer 122a can be configured to receive an input image 401 and to provide an output image 402.

The image enhancer 122a can be coupled to an output of the endoscope camera 134a, such as by receiving an image frame from the endoscope transmitter 135 as an input image 401. In one embodiment, the image frame is received by electromagnetic transmission from the endoscope transmitter 135; however, the image frame can be received by any other technique for data transfer.

The input image 401 can include a 400×400×3 image (400 pixels wide by 400 pixels high, with three channels). In one embodiment, the channels include colors such as red, green, and blue (RGB).

In alternative embodiments, image data provided by the imaging sensor 134a can have a resolution of about 640×480×3 (that is, 640 pixels wide by 480 pixels high, with three channels). However, it might occur that image contrast and/or frame rate provided by the image sensor 134a at that resolution is not preferred, such as because collecting image data at that image contrast and/or frame rate does not provide an adequate amount of contrast. A subsampled mode available using the imaging sensor 134a can be used to provide an image with a resolution of about 320×320×3 image data (that is, 320 pixels wide by 320 pixels high). The image data received from the subsampled mode can be cropped to about 240×240×3 (that is, 240 pixels wide by 240 pixels high), such as might match an aspect ratio of a colonoscope, before undoing distortion and upscaling.

While this Application primarily describes methods and systems using particular selected imaging sensors 134a and particular selected image resolutions, in the context of the invention, there is no particular requirement for any such limitation. After reading this Application, those skilled in the art would recognize that other selected image resolutions, and methods and systems adapted thereto, would be workable, and are within the scope and spirit of the invention. Similarly, while this Application primarily describes methods and systems using an RGB color gamut to describe particular selected colors for imaging, in the context of the invention, there is no particular requirement for any such limitation. After reading this Application, those skilled in the art would recognize that other color gamuts and color encodings, and methods and systems adapted thereto, would be workable, and are within the scope and spirit of the invention.

One issue with respect to the input image 401 is that it is responsive to an image generated by the endoscope imaging sensor 134a. The image generated by the endoscope imaging sensor 134a might be relatively low-resolution and might be distorted in one or more ways. For one example, an output from the endoscope imaging sensor 134a might include one or more geometric distortions such as barrel distortion and possibly other types of linear or nonlinear distortion. For another example, an output from the endoscope imaging sensor 134a might also include one or more lighting distortions, such as the image being darker at its edges. Other distortions are possible in response to conditions within the colon where the endoscope imaging sensor 134a is operated. For another example, distortions that relate to colors or color gradients are also possible, such as possibly distortion of colors in low light conditions or other color or color gradient distortions.

In one embodiment, the image enhancer 122a can include an enhance model 410, a transfer element 420, an upscale model 430, and an output element 440.

### Enhance Model

The input 401 can be coupled to the enhance model 410.

The enhance model 410 can include an enhance element 411, a first encoder/decoder stage 412, a second encoder/decoder stage 413, a third encoder/decoder stage 414, a bottleneck transition stage 415, and a final convolution stage 416. These stages can be configured using a configuration similar to a UNet technique.

As described herein, the enhance model 410 can receive the input image 401. The input image 401 can include a 400×400×3 image (400 pixels wide by 400 pixels high, with three channels). In one embodiment, the channels include colors such as red, green, and blue (RGB).

As described above, in alternative embodiments, the input 401 can have a different size or resolution, such as an image that is 240×240×3 (240 pixels wide by 240 pixels high, with three channels). In such cases, the image enhancer 122a would operate similarly, with each image described herein having a number of associated pixels and channels that depend on the input image 401. For example, where the first encoder/decoder stage 412 is described herein as providing a 200×200 image from a 400×400 image, the first encoder/decoder stage 412 would instead provide a 120×120 image from a 240×240 image. Other image sizes as described herein would similarly have a number of associated pixels and channels that depend on the input image 401. After reading this Application, those skilled in the art would recognize the relation between a size of the input image 401 and a size of any particular intermediate image and would easily be able to interpret the description herein in the context of a different size for the input image.
Enhance Element. The enhance model 410 can include an enhance element 411 configured to add information (such as geometric coordinates) to the input image 401. For example, for each pixel, the enhance model 410 can add an X coordinate and a Y coordinate, where those coordinates are associated with a position of that pixel in the image. This can be helpful at later processing stages when attempting to correct for one or more types of distortion responsive to position in the image. For example, as described herein, the image might be darker or otherwise have lower resolution at its edges.

After adding coordinates with respect to a 2-dimensional (2D) field, the enhanced image provided by the enhance element 411 can be 400×400×5. For example, the 400×400×5 image can include a 400×400 array of pixels, three channels for RGB colors, and two additional channels for X and Y geometric coordinates. The 400×400×5 image can be coupled to the first encoder/decoder stage 412.

While this Application primarily describes operation with respect to images having a 2D image field, in the context of the invention, there is no particular requirement for any such limitation. After reading this Application, those skilled in the art would recognize that many alternatives would be workable and are within the scope and spirit of the invention. For a first example, the image field can be configured or disposed as a 3-dimensional (3D) field, such as having depth and/or another 3D imaging effect. For a second example, the image field can be configured or disposed as a “2½-dimensional” (2½D) field, such as having a two dimensions of location and a “half” dimension of limited scope representing some further aspect of the body structure of medical effect being imaged. Those skilled in the art would also recognize that other and further possible alternatives are also workable and are within the scope and spirit of the invention.

Further, while this Application primarily describes operation with respect to still images, or with respect to individual frames of a multi-frame video image (such as a sequence of frames illustrating a moving picture or other encoding of a moving video image), in the context of the invention, there is no particular requirement for any such limitation. After reading this Application, those skilled in the art would recognize that many alternatives would be workable and are within the scope and spirit of the invention. For a first example, a sequence of images received from the image enhancer can operate with respect to a 3-dimensional (3D) field, such as having depth and/or another 3D imaging effect, whether in combination with a moving image effect or otherwise. For a second example, the image field can be configured or disposed with at least some of the representation encoded by the image being time-varying (such as a sequence of medical data or other information of interest to medical personnel), and with some of the representation encoded by the image being substantially static. Those skilled in the art would also recognize that other and further possible alternatives are also workable and are within the scope and spirit of the invention.
First encoder. The first encoder/decoder stage 412 can include a first encoder portion, including a first encoder element 412a and a first maxpool element 412b, and a first skip data path 412c, and a first decoder portion, including a first upsample element 412d and a first decoder/skip element 412e.

The first encoder element 412a can include a convolutional neural network configured to perform convolution on each set of pixels. For example, the first encoder element 412a can perform convolution on a 3×3 window centered on each pixel, and can select a set of 64 image features associated with each such pixel. This can generate a 400×400×64 image.

The first maxpool element 412b can be configured to receive the 400×400×64 image and to select one set of features from each 2×2 set of pixels. In one embodiment, the first maxpool element 412b can select a maximum value for each such feature. This can generate a 200×200×64 image, thus, an image with 64 features for each pixel but only 200×200 pixels in size.

The first skip data path 412c can be configured to provide the 400×400×64 image to the first decoder/skip element 412e, which can perform its operations later in the process performed by the enhance model 410.

In one embodiment, the first encoder/decoder stage 412 can include a double convolutional block, thus performing two convolutions, batch normalizations, and nonlinearizations per stage. Each nonlinearization can include an activation function, such as might be performed with respect to a neural network.

In alternative embodiments, the first encoder/decoder stage 412 can perform one, or three, or a different number of convolutions, batch normalizations, and nonlinearizations per stage. Similarly, the second encoder/decoder stage 413 and the third encoder/decoder stage 414 can each include two (or a different number of) convolutions, batch normalizations, and nonlinearizations per stage. In the context of the invention, there is no particular requirement that each of the first, second, and third encoder/decoder stage are required to perform the same number of convolutions, batch normalizations, and nonlinearizations.

In alternative embodiments, the enhance model 410 (and other neural networks described herein) might alternatively use other functions instead or in lieu of batch normalization. For example, where batch normalization is referred to herein, the enhance model 410 (and other neural networks) can use another norm, such as a GroupNorm function or another function. Similarly, in alternative embodiments, the enhance model 410 (and other neural networks described herein) might alternatively use other nonlinear activation functions, such as Gaussian Error Linear Unit (GELU), LeakyReLU, softmax, or another function.
Second encoder. The second encoder/decoder stage 413 can include a second encoder portion, including a second encoder element 413a and a second maxpool element 413b, and a second skip data path 413c, and a second decoder portion, including a second upsample element 413d and a second decoder/skip element 413e.

The second encoder element 413a can include a convolutional neural network configured to perform convolution on each set of pixels. For example, the second encoder element 413a can perform convolution on a 3×3 portion of the image centered on each pixel, and can select a set of 128 image features associated with each such pixel. This can generate a 200×200×128 image.

The second maxpool element 413b can be configured to receive the 200×200×128 image and to select one set of features from each 2×2 set of pixels. In one embodiment, the second maxpool element 413b can select a maximum value for each such feature. This can generate a 100×100×128 image, thus, an image with 128 features for each pixel but only 100×100 pixels in size.

The second skip data path 413c can be configured to provide the 200×200×128 image to the second decoder/skip element 413e, which can perform its operations later in the process performed by the enhance model 410.
Third encoder. The third encoder/decoder stage 414 can include a third encoder portion, including a third encoder element 414a and a third maxpool element 414b, and a third skip data path 414c, and a third decoder portion, including a third upsample element 414d and a third decoder/skip element 414e.

The third encoder element 414a can include a convolutional neural network configured to perform convolution on each set of pixels. For example, the third encoder element 414a can perform convolution on a 3×3 portion of the image centered on each pixel, and can select a set of 256 image features associated with each such pixel. This can generate a 100×100×256 image.

The third maxpool element 414b can be configured to receive the 100×100×256 image and to select one set of features from each 2×2 set of pixels. In one embodiment, the third maxpool element 414b can select a maximum value for each such feature. This can generate a 50×50×256 image, thus, an image with 256 features for each pixel but only 50×50 pixels in size.

The third skip data path 414c can be configured to provide the 100×100×256 image to the third decoder/skip element 414e, which can perform its operations later in the process performed by the enhance model 410.

In one embodiment, the enhance model 410 can be configured to use between 2-4 such encoder/decoder stages. While this Application primarily describes an enhance model 410 with three such encoder/decoder stages, in the context of the invention, there is no particular requirement for any such limitation; other numbers of such stages would be workable and are within the scope and spirit of the invention.
Bottleneck transition stage. The bottleneck transition stage 415 can include a convolutional neural network configured to convolve the 50×50×256 image and to provide a 50×50 image with 512 features, thus, a 50×50×512 image.
Third decoder. The third upsample element 414d can include a convolutional neural network configured to upsample the image back from 50×50×512 to 100×100×256.

The third decoder/skip element 414e can include a convolutional neural network configured to retrieve information from the third skip data path 414c and to add that information back into the image it generates. This can provide a 100×100×256 image with sufficient detail for later stages.
Second decoder. The second upsample element 413d can include a convolutional neural network configured to upsample the image back from 100×100×256 to 200×200×128.

The second decoder/skip element 413e can include a convolutional neural network configured to retrieve information from the second skip data path 413c and to add that information back into the image it generates. This can provide a 200×200×128 image with sufficient detail for later stages.
First decoder. The first upsample element 412d can include a convolutional neural network configured to upsample the image back from 200×200×128 to 400×400×64.

The first decoder/skip element 412e can include a convolutional neural network configured to retrieve information from the first skip data path 412c and to add that information back into the image it generates. This can provide a 400×400×64 image with sufficient detail for later stages.
Final convolution stage. The final convolution stage 416 can include a convolutional neural network configured to select three channels from the 400×400×64 image. In one embodiment, the three selected channels include RGB color channels. This can provide a 400×400×3 image suitable for the upscale model 430.

### Transfer Element

The Enhance Model 410 Can Be Coupled to a Transfer Element 420.

In one embodiment, the transfer element 420 can include an enhanced sigmoid element configured to limit the pixel values in the 400×400×3 image to within the proper data range for each pixel. For example, the enhanced sigmoid element can include a sigmoid function configured to limit each pixel to a color value of between 0 and 1, such as expressed as an 8-bit value between 0 and 255.

### Upscale Model

In an alternative embodiment in which the input image is initially 240×240 (instead of 400×400, as might occur as described above), the enhance model 410 can be configured to provide a 240×240 image for coupling to the transfer element 420. In such case, the transfer element 420 can be configured to provide a 240×240 image to the upscale model 430, and the upscale model 430 can be configured to accept a 240×240 image.

The transfer element 420 can be coupled to an upscale model 430.

The upscale model 430 can include a resizing element 431, a first convolution element 432, a second convolution element 433, a third convolution element 434, a fourth convolution element 435, and a pixelshuffle element 436.
Resizing element. The resizing element 431 can be configured to resize the image from 400×400 to 540×540, such as using a bicubic resizing technique. In one embodiment, the same three channels (RGB colors) are preserved.

In an alternative embodiment in which the input image is initially 240×240 (instead of 400×400, as might occur as described above), the upscale model 430 can be configured to accept a 240×240 image. In such cases, the resizing element 431 can be configured to resize the image from 240×240 to 270×270 (or another size for which 1080×1080 is an integer multiple) and the upscale model 430 can be configured to operate on 270×270 images (or another size) until it is ready to upscale to 1080×1080.
First convolution element. The first convolution element 432 can include a convolutional neural network configured to perform convolution on the image with respect to a 5×5 convolution window, and can select a set of 160 image features associated with each such pixel. The first convolution element 432 can also perform batch normalization on each such convolution window and can nonlinearize a result thereof using a ReLU activation function. This can provide a 540×540×160 image.
Second convolution element. The second convolution element 433 can include a convolutional neural network configured to perform convolution on the image with respect to a 3×3 convolution window, and can select a set of 80 image features associated with each such pixel. The second convolution element 433 can also perform batch normalization on each such convolution window and can nonlinearize a result thereof using a ReLU activation function. This can provide a 540×540×80 image.
Third convolution element. The third convolution element 434 can include a convolutional neural network configured to perform convolution on the image with respect to a 3×3 convolution window, and can select a set of 40 image features associated with each such pixel. The third convolution element 434 can also perform batch normalization on each such convolution window and can nonlinearize a result thereof using a ReLU activation function. This can provide a 540×540×40 image.
Fourth convolution element. The fourth convolution element 435 can include a convolutional neural network configured to perform convolution on the image with respect to a 3×3 convolution window, and can select a set of 12 image features associated with each such pixel. This can provide a 540×540×12 image.
Pixelshuffle element. The pixelshuffle element 436 can be configured to provide a 1080×1080×3 image, having three color channels. In one embodiment, for each location within the 540×540×12 image, the pixelshuffle element 436 collects the 12 input channels for each such pixel and apportions them to 3 output channels (RGB colors) for each of 4 output pixels.

In an alternative embodiment in which the input image is initially 240×240 (instead of 400×400, as might occur as described above), the upscale model 430 can be configured to accept a 270×270 image. In such cases, the pixelshuffle element 436 can be configured to resize the image from 270×270 (or another size for which 1080×1080 is an integer multiple) to 1080×1080, collecting 48 input channels for each such pixel and apportioning them to 3 output channels for each of the 16 output pixels. In such alternative embodiments, because the output image is n x n times the size of the resized image, the pixelshuffle element 436 would collect 3n² input channels for each such pixel and apportion them to 3 output channels for each of the n² output pixels.

### Output Element

The upscale model 430 can be coupled to an output element 440.

In one embodiment, the output element 440 can include an output sigmoid element.

In one embodiment, the output sigmoid element can be configured to limit the pixel values in the 1080 ×1080×3 image to within the proper data range for each pixel, similar to the transfer element 420. For example, the output sigmoid element can include a sigmoid function configured to limit each pixel to a color value of between 0 and 1, such as expressed as an 8-bit value between 0 and 255.

The output element 440 can be coupled to an output of the image enhancer 122a and can provide the output image 402.

### Preparing for Upscaling

In one embodiment, the system 100 can be configured to perform pre-processing, in which the image enhancer 122a is configured to prepare it to enhance images and to upscale those images, consistent with a set of real images (sometimes referred to herein as “ground truth”) sourced from medical imaging of actual medical structures.

In one embodiment, the ground truth includes a set of images selected or derived from the “REAL-Colon” dataset, such as described at Biffi et al., “REAL-Colon: A dataset for developing real-world AI applications in colonoscopy”, Scientific Data 11:539 (2024), available at www. nature. com/articles/s 41597-024-03359-0, including about 2.7 million images, each 1080×1080×3, from about 60 examples of medical procedures (such as colonoscopies), collected at about 30 frames per second, collectively about 30,000 to 100,000 individual frames. While this description primarily relates to using this particular set of images, in the context of the invention there is no particular requirement for any such limitation; any reasonable dataset that could be used with the methods and systems described herein would be workable, and the use of any reasonably similar dataset is within the scope and spirit of the invention.
Image degradation simulator. In one embodiment, an image degradation simulator 450 can be configured to receive a ground truth image of an internal medical structure, such as a portion of a colon or a structure therein, or another medical structure, and to generate a degraded image. The degraded image can be coupled to the image enhancer 122a as its input image 401.

In one embodiment, an image degradation simulator 450 can be configured to perform realistic distortions to the ground truth images, such as distortions that might be introduced by a real world camera collecting real world images from inside the body or otherwise in circumstances in which distortion might be introduced.

For a first example, the image degradation simulator 450 can be configured to perform distortion using one or more of the following parameters:
barrel distortion, such as with radial distortion=about −0.203, higher-order radial distortion=about 0.434, and effective focal length=about 788.76;
camera matrix construction using principal point centering; and
OpenCV-based distortion application for geometric accuracy.

For a second example, the image degradation simulator 450 can also be configured to perform distortion using one or more of the following parameters:
vignetting simulation using radially-varying intensity falloff, such as with vignetting strength=about 0.668;
gaussian-based distance calculation from an image center; and
power-law falloff with an exponent=about 1.5, for realistic edge darkening.

For a third example, the image degradation simulator 450 can also be configured to perform distortion using one or more of the following parameters:
color channel response simulation, such as with an R-channel scale=about 1.234, a G-channel scale=about 0.739, and a B-channel scale=about 0.712.

For a fourth example, the image degradation simulator 450 can also be configured to perform distortion using one or more of the following parameters:
brightness boosting, such as with a center-weighted gaussian parameter=about 72.33.

For a fifth example, the image degradation simulator 450 can also be configured to perform distortion using one or more of the following parameters:
downsampling from 1080×1080 to 400×400;
Gaussian noise and JPEG compression; and
stochastic parameter variation.

In one embodiment, the image degradation simulator 450 can be configured to provide distortions associated with a particular one or more cameras or other image-collection devices likely to be used in associated with the image enhancer 122a. For example, the image degradation simulator 450 can be configured to provide distortions associated with the OVM6946 colonoscopy camera, or an equivalent thereof. While this description primarily relates to using these particular types of distortions and parameters therefore, in the context of the invention there is no particular requirement for any such limitation; any reasonable set of distortions and parameters that could be used with the methods and systems described herein would be workable, and the use of any reasonably similar dataset is within the scope and spirit of the invention.

In one embodiment, the image enhancer 122a can be coupled to the image degradation simulator 450, so as to receive a set of input images 401 there-from. In response to those input images 401, the image enhancer 122a can be configured to perform upscaling with respect to those input images 401 provided by the image degradation simulator 450 and to provide a set of output images 402 in response thereto. This can allow the image enhancer 122a, including its enhance model 410 and its upscale model 430, to be configured to undo the simulated degradation that was performed by the image degradation simulator 450.

In one embodiment, the output images 402 can be compared with the ground truth, so as to determine a measure of quality for operation of the image enhancer 122a. In response to that measure of quality, parameters associated with the image enhancer 122a can be adjusted or otherwise configured to improve that measure of quality, that is, to adjust the image enhancer 122a so that it can reliably undo the simulated degradation that was performed by the image degradation simulator 450.

In one embodiment, the measure of quality can be responsive to one or more loss functions. For example, the loss function can include one or more of the following: MAE (mean absolute error), PSNR (peak signal to noise ratio), MS-SSIM (multiscale structural similarity), LPIPS (learned perceptual image patch similarity), or another loss function. In one embodiment, the composition of the enhance model 410 and the upscale model 430 is able to achieve an end-to-end PSNR of about 29 with respect to undoing distortion introduced by the image degradation simulator 450.

In one embodiment, the enhance model 410 and the upscale model 430 can each include one or more artificial neural networks, as described herein. Each such artificial neural network can be configured to using a set of weights associated with each particular artificial neuron. For example, the artificial neural networks can each be configured using training techniques adapted for adjusting weights in neural networks, such as gradient descent or other backpropagation techniques, or such as alternative techniques for selecting or adjusting those weights.
Preparing enhance model. In one embodiment, the enhance model 410 can include a neural network similar to the modified UNet structure that is described herein. The enhance model 410 can be configured to be coordinate-aware, that is, to be able to respond to and correct image distortion in response to X and Y coordinates of each pixel and thus to distortions that are position-dependent.

In one embodiment, the enhance model 410 can be adjusted independently from the upscale model 430. For example, the image degradation simulator 450 can be configured to provide an input image 401 that has been degraded to introduce distortions, and an output of the enhance model 410 can be compared with a downsampled version of that input image 401. Thus, the enhance model 410 can be configured to undo just the distortions and not the downsampling introduced by the image degradation simulator 450.

In one embodiment, the enhance model 410 can also be adjusted with portions thereof independently held constant. For example, the enhance model 410 can have each of its first skip data path 412c, second skip data path 413c, or third skip data path 414c, independently operational or suppressed. This can allow the enhance model 410 to be adjusted with relatively coarser or finer adjustment of its attempt to undo distortions.
Preparing upscale model. In one embodiment, the upscale model 430 can include a neural network similar to the modified ESPCN (efficient subpixel convolutional neural network) structure that is described herein. As described herein, the upscale model 430 can perform multistage upscaling of resolution, such as from 400×400 to 540×540 to 1080×1080.

In one embodiment, the upscale model 430 can be adjusted independently from the enhance model 410. For example, the image degradation simulator 450 can be configured to provide an input image 401 that has been downscaled without distortions, and an output of the upscale model 430 can be compared with the ground truth image. Thus, the upscale model 430 to be configured to undo just the downsampling introduced by the image degradation simulator 450.

In one embodiment, the enhance model 410 and the upscale model 430 can also be adjusted in combination. For example, once the enhance model 410 and the upscale model 430 are each independently adjusted, they can be further adjusted in combination so as to perform fine-tuned adjustment.

### ALTERNATIVE EMBODIMENTS

While this Application primarily describes a system, and techniques for use therewith, related to performing specific medical procedures such as colonoscopies, there is no particular requirement for any such limitation, and there is no need for any such restriction. After reading this Application, those skilled in the art will recognize that the techniques described herein are applicable to other and varied procedures, to be performed inside the body, other and varied procedures involving retrieving and presenting imaging data, or otherwise. For one example, the system and methods can be disposed to retrieve and present imaging data from within blood vessels or a cardiac or pulmonary subsystem. For another example, the system and methods can be disposed to retrieve and present imaging data when that imaging data is transmissible at a relatively low data rate or resolution (such as in an environment having substantial electromagnetic noise) and to be presented at a relatively high resolution.

While this Application primarily describes a system, and techniques for use therewith, related to preparing and operating an image enhancer using artificial neural networks, there is no particular requirement for any such limitation, and there is no need for any such restriction. After reading this Application, those skilled in the art will recognize that the techniques described herein are applicable to other and varied procedures, to be performed with respect to image enhancement using artificial intelligence or machine learning techniques, or other techniques, and other and varied procedures involving preparing to enhance imaging data, or otherwise.

This Application describes a preferred embodiment with preferred process steps and, where applicable, preferred data structures. After reading this Application, those skilled in the art would recognize that, where any calculation or computation is appropriate, embodiments of the description can be implemented using general purpose computing devices or switching processors, special purpose computing devices or switching processors, other circuits adapted to particular process steps and data structures described herein, or combinations or conjunctions thereof, and that implementation of the process steps and data structures described herein would not require undue experimentation or further invention.

The claims are incorporated into the specification as if fully set forth herein.

1. An imaging system including an endoscope accessory,
the endoscope accessory being configured to be fitted to an endoscope, the endoscope including a first camera;
the endoscope accessory including a second camera disposed with a field of view substantially distinct from the first camera;
the endoscope accessory being disposed to transmit wireless signals including image data from inside a patient's body;
wherein a display system coupled to the wireless signals is disposed to select between image data from the first camera and image data from the second camera, in response to medical personnel.

2. An imaging system as in claim 1, wherein
at least one of the first camera or the second camera is configured to transmit the image data to present a substantially real-time representation, whether using a sequence of still images or using a video image,
wherein the substantially real-time representation provides for relatively smooth image perception by medical personnel performing the medical procedure.

3. An imaging system as in claim 1, including
a receiver outside the patient's body couplable to image data received from inside a patient's body;
an image enhancer configured to upscale the image data to a higher resolution than available for transmission from inside the body;
wherein the higher resolution is sufficient for medical personnel to identify and treat medical conditions within the patient.

4. An imaging system as in claim 3, wherein
the image enhancer uses an artificial intelligence or machine learning technique to upscale the image data, wherein the artificial intelligence or machine learning technique has been adjusted, configured, or disposed to use:
a first enhancement to undo a distortion induced by capture of the image data, the first enhancement being adjustable in response to a distorted image,
a second enhancement to upscale an image, the second enhancement being adjustable in response to a downsampled image,
the first enhancement and the second enhancement being independently configurable.

5. An imaging system as in claim 1, wherein
at least a portion of the image data includes a three-dimensional (3D) effect.

6. An imaging system as in claim 1, wherein
at least one of the first camera or the second camera is configured to transmit image data from inside a patient's body with a relatively smooth image, whether in a sequence of still images or in a video image, which does not interfere with perception by medical personnel performing the medical procedure.

7. An imaging system as in claim 6, wherein
at least one of the first camera or the second camera is disposed to transmit image data disposed to present a substantially real-time representation, whether using a sequence of still images or using a video image,
wherein the substantially real-time representation provides for relatively smooth image perception by medical personnel performing the medical procedure.

8. An imaging system, including
a receiver outside the patient's body couplable to image data received from inside a patient's body;
an image enhancer configured to upscale the image data to a higher resolution than available for transmission from inside the body;
wherein the higher resolution is sufficient for medical personnel to identify and treat medical conditions within the patient.

9. An imaging system as in claim 8, including
a transmitter disposed to transmit wireless signals including image data from inside a patient's body;
wherein a display system coupled to the wireless signals is disposed to upscale the image data for presentation to medical personnel, the upscaled image data being higher resolution than available for transmission from inside the body.

10. A method, including steps of
coupling a receiver outside a patient's body to image data received from inside a patient's body;
upscaling the image data to a higher resolution than available for transmission from inside the body;
wherein the higher resolution is sufficient for medical personnel to identify and treat medical conditions within the patient.

11. A method as in claim 10, including steps of
to an endoscope having a first camera, coupling a second camera having a field of view substantially distinct from the first camera;
wherein the steps of coupling include transmitting wireless signals including image data from inside a patient's body;
in a display system coupled to the wireless signals, selecting between image data from the first camera and image data from the second camera, in response to medical personnel.

12. A method as in claim 10,
wherein the steps of coupling and upscaling include transmitting image data from inside a patient's body with a relatively smooth image, whether in a sequence of still images or in a video image, without interfering with perception by medical personnel performing the medical procedure.

13. A method as in claim 10,
wherein the steps of coupling include transmitting wireless signals including image data from inside a patient's body;
and including steps of
in a display system coupled to the wireless signals, upscaling the image data for presentation to medical personnel, upscaled image data being higher resolution than available for transmission from inside the body.

14. An imaging system as in claim 8, wherein
the image enhancer is configured to undo a distortion induced by capture of the image data.

15. An imaging system as in claim 8, wherein
the image enhancer includes a first enhancer element configured to undo a distortion induced by capture of the image data and a second enhancer element configured to upscale an image.

16. An imaging system as in claim 8, wherein
the image enhancer includes a first enhancer element disposed to undo a distortion induced by capture of the image data, the first enhancer element being configured in response to a distorted image;
the image enhancer includes a second enhancer element disposed to upscale an image, the second enhancer element being configured in response to a downsampled image;
the first enhancer element and the second enhancer element being independently configurable.

17. An imaging system as in claim 16, wherein
the image enhancer is configurable in response to a combined operation of the first enhancer element and the second enhancer element.

18. A method as in claim 10, including steps of
undoing a distortion induced by capture of the image data.

19. A method as in claim 10, including steps of
performing a first enhancement to undo a distortion induced by capture of the image data, the first enhancement being adjustable in response to a distorted image;
performing a second enhancement to upscale an image, the second enhancement being adjustable in response to a downsampled image;
the first enhancement and the second enhancement being independently configurable.

20. A method as in claim 19, wherein
the first enhancement and the second enhancement are each configurable in response to a combined operation of the first enhancement and the second enhancement.
